(19) United States
(12) Patent Application Publication
(10) Pub. No.: US 2022/0364122 A1
(43) Pub. Date: Nov. 17, 2022

(54) BACTERIAL PLATFORM FOR DELIVERY OF GENE-EDITING SYSTEMS TO EUKARYOTIC CELLS

(71) Applicant: SiVEC Biotechnologies LLC, Fort Collins, CA (US)

(72) Inventors: Lyndsey M. Linke, Fort Collins, CO (US); Darcy Mora, Fort Collins, CO (US)

(73) Assignee: SiVEC Biotechnologies LLC, Fort Collins, CO (US)

(21) Appl. No.: 17/595,993

(22) PCT Filed: Jun. 1, 2020
(86) PCT No.: PCT/US20/35613

§ 371 Date: Dec. 1, 2021

(51) Int. Cl. C12N 15/90; C12N 15/11; C12N 9/22; C12N 15/70; C07K 14/245

(52) U.S. Cl. CPC C12N 15/907; C12N 15/11; C12N 9/22; C12N 15/70; C07K 14/245; C12N 2310/20; C12N 2800/80; C12N 2800/101

(57) ABSTRACT

A bacterial-mediated gene-editing delivery platform that uses invasive, non-pathogenic bacteria to deliver gene-editing cargo, including CRISPR/Cas systems, to eukaryotic cells. The bacteria contain a prokaryotic expression cassette encoding the gene-editing cargo.

### CROSS-REFERENCE TO RELATED APPLICATIONS

This application claims the benefit of U.S. Provisional Application No. 62/856,055, filed Jun. 1, 2019.

### FIELD OF INVENTION

This invention relates to the delivery of gene-editing systems to eukaryotic cells using a non-pathogenic bacterial delivery platform.

### BACKGROUND OF THE INVENTION

Gene-editing is a form of genetic engineering in which DNA is inserted, deleted, modified or replaced in the genome of a living organism. The editing of specific sequences within a genome can be used for research purposes, as well as in gene therapy by targeting mutations to specific genes or inserting a functional gene into an organism and targeting it to replace the defective one that is associated with a pathogenic phenotype or genetic disease. The common approach to gene-editing is to use engineered or synthetic nucleases. One example is CRISPR (clustered regularly interspaced short palindromic repeat)/Cas or the CRISPR/Cas system, which is rapidly advancing the field of genome editing.

### SUMMARY OF THE INVENTION

The present invention provides a bacterial-mediated gene-editing delivery platform that uses invasive, non-pathogenic bacteria to deliver gene-editing cargo, including but not limited to CRISPR/Cas systems, to eukaryotic cells, wherein the bacteria contain a prokaryotic promotor associated with a prokaryotic expression cassette encoding the gene-editing cargo.

In one aspect the present invention provides a method for generating and delivering gene-editing systems, including nuclease-based gene-editing systems, to eukaryotic cells. The method includes the step of invading the eukaryotic cells with invasive non-pathogenic bacteria. The bacteria contain a plasmid having a prokaryotic expression cassette encoding a gene-editing system. The prokaryotic expression cassette can operate under the control of one or more prokaryotic promoters, such as the T7, lacUV5, gapA, T5, recA, Ptac, Patac, pA1, lac, Sp6, araBad, trp, and hybrid promoters. These prokaryotic promoters can be inducible, constitutive or a combination thereof. It is contemplated that, where there are multiple promoters on a plasmid or multiple plasmids in a bacterium, different promoter types can be used based upon the relative activity of the promoter in the system. By using different types of prokaryotic promoters, transcription can be tailored to control levels of transcripts. By using prokaryotic promoters in the bacterium, transcription of the gene editing system components occurs within the bacterium and not the target eukaryotic cell.

In a second aspect the present invention provides a composition comprising a bacterium for modifying the genome of a eukaryotic cell. The bacterium has a prokaryotic vector. The prokaryotic vector (or vectors) can be a plasmid, or multiple plasmids, employing prokaryotic promoters to control transcription from the plasmid. The plasmid or plasmids can encode a CRISPR/Cas system having (a) at least one CRISPR associated (Cas) enzyme, (b) at least one tracr sequence(s), and (c) at least one CRISPR RNA (crRNA). The crRNA can have (i) at least one CRISPR direct repeat region and (ii) at least one spacer sequence capable of hybridizing to a target sequence in a eukaryotic cell. The plasmid encoding a CRISPR/Cas system can optionally include a nuclear localization signaling sequence to facilitate nuclear translocation of the gene-editing system to the nuclei of eukaryotic cell. The bacterium is engineered to be invasive to the eukaryotic cell. In this second aspect of the present invention, the bacteria generate a programmable nuclease system to direct CRISPR complex activity in the nuclei of eukaryotic cells.

In a third aspect, the present invention provides a composition comprising the use of a bacterial delivery system to generate and deliver a gene-editing system (gene-editing cargo), capable of entering a eukaryotic cell using the invasion factors, invasin protein (encoded by the inv gene) and the listeriolysin O (LLO) protein (encoded by the hlyA gene). The bacterial system is engineered to generate and/or deliver the gene-editing system to a eukaryotic cell. The composition comprising the use of a bacterial cell to generate and/or deliver a gene-editing system may contain one or more prokaryotic plasmid with one or more prokaryotic promoter, one or more eukaryotic plasmid with one or more eukaryotic promoter, or any combination thereof. In this third aspect of the present invention, the bacterial system utilized is capable of attaching to eukaryotic cells by expressing the invasin protein and escaping the eukaryotic endosome by expressing the LLO protein, thereby allowing for the gene-editing cargo to be released into the cytoplasm of the eukaryotic cell and where appropriate, translocate to the nuclei of the eukaryotic cell.

In a fourth aspect, the present invention provides a composition for in vitro, in vivo, or ex vivo engineering of eukaryotic cells to insert, remove or replace a gene. For in vitro use, the bacterial system engineered to generate and deliver the gene-editing system, can be used to insert, remove or replace a region of interest on a eukaryotic cell genome and modify the target DNA in vitro. For example, the CRISPR motifs or PAM sequences present on the target DNA in vitro (in living cells outside of a living system) can be recognized by the CRISPR/Cas gene-editing system generated from a prokaryotic plasmid and delivered to the eukaryotic cell by the bacterial cell. In another embodiment, the CRISPR motifs or PAM sequences on the target DNA in vitro are recognized by the CRISPR/Cas gene-editing system that is transcribed and translated from a eukaryotic plasmid inside the targeted eukaryotic cell following delivery by the bacterial cell. These genome modification experiments can be done in vitro, including but not limited for use with primary cells, immortalized cells, dividing, and non-dividing cells, and hematopoietic stem/progenitor cells. These in vitro gene-editing applications can include but are not limited to the purpose of studying disease, pathogenesis of a particular gene modification, infectious agents, genetic disorders, functional genome screening, chromosome painting, constructing disease models (including cancer models), for use with in vitro selection libraries, mismatch-detection nuclease assays, high-throughput profiling assays, diagnostics, and related next-generation sequencing activities. For ex vivo genome editing and modification, eukaryotic cells are isolated from a living system and the bacterial system engineered to generate and deliver the gene-editing system is used to insert, remove, or replace a region of interest on the isolated eukaryotic cell genome and modify the target DNA ex vivo. For example, the CRISPR motifs or PAM sequences present ex vivo (in cells isolated from a living system) can be recognized by the CRISPR/Cas gene-editing system generated from a prokaryotic plasmid and delivered to the eukaryotic cell, ex vivo, by the bacterial cell. In another embodiment, the CRISPR motifs or PAM sequences present on the DNA ex vivo is recognized by the CRISPR/Cas gene-editing system that is transcribed and translated from a eukaryotic plasmid inside the targeted eukaryotic cell following delivery by the bacterial cell. In some cases, this invention is used to achieve a therapeutic success whereby the isolated cells are treated and returned to the target tissue for transplantation back into the living system from which the eukaryotic cells came. An example of an ex vivo (or in vitro) application is for chimeric antigen receptor (CAR) T cell therapy, in which T cells derived from patient blood are extracted and the genome of the isolated T cells are edited to express artificial receptors targeted to a specific tumor antigen using the present invention. For in vivo genome editing and modification, the bacterial system engineered to generate and deliver the gene-editing system can be used to insert, remove, or replace a region of interest on a eukaryotic cell genome for a specific genome modification of DNA in a living system. In some cases, this invention is used to deliver the gene-editing system to a targeted tissue (such as the eye, muscle, lungs, brain, nasal cavity, gastrointestinal tract, heart, skin, oral cavity, genital tissue, or any combination thereof). For example, the CRISPR motifs or PAM sequences present in vivo can be recognized by the CRISPR/Cas gene-editing system generated from a prokaryotic plasmid and delivered to the targeted eukaryotic cell, in vivo, by the bacterial cell. In another embodiment, the CRISPR motifs or the PAM sequences present on the DNA in vivo is recognized by the CRISPR/Cas gene-editing system that is transcribed and translated from a eukaryotic plasmid inside the targeted eukaryotic cell following delivery by the bacterial cell to a particular tissue. These in vivo gene-editing applications can include but are not limited to the purpose of studying disease, pathogenesis of a particular gene modification, infectious agents, genetic disorders, developing gene knock-in and knock-out animals, including the generation of transgenic embryos, for treating disease, preventing disease, and treating rare genetic disorders.

In a fifth aspect, the present invention provides a composition comprising the use of a bacterial delivery system to generate and deliver a gene-editing system (gene-editing cargo), in which the elements that comprise the gene-editing system are expressed from a recombinant plasmid, expressed from the bacterial chromosome (integrated into the bacterial chromosome), or expressed using any combination thereof Similarly, the invasion factors (inv and hlyA) required to enter a eukaryotic cell and escape the eukaryotic cell endosome are expressed from a recombinant plasmid, expressed from the bacterial chromosome, or expressed using any combination thereof. Any of the components of the gene-editing system and any of the factors required for bacterial invasion can be expressed in combination from one or more recombinant plasmids, from the chromosome of the bacteria, or any combination thereof.

In a sixth aspect, the present invention provides a plasmid that contains one or more of a Cas gene, an NLS, a CRISPR direct repeat region, a spacer sequence (or an insertion site for a spacer sequence) and a tracr sequence. These elements can be expressed from the plasmid, such as by one or more prokaryotic promoters upon transformation of a bacterial cell. In an advantageous embodiment the plasmid contains a spacer sequence (or an insertion site for a spacer sequence) and the prokaryotic or eukaryotic promoter.

### BRIEF DESCRIPTION OF THE DRAWINGS

For a fuller understanding of the invention, reference should be made to the following detailed description, taken in connection with the accompanying drawings, in which:

FIG. 1 is an illustration providing a graphical representation of the bacterial mediated gene-editing delivery system that uses invasive, non-pathogenic bacteria to deliver gene-editing cargo, such as a CRISPR/Cas systems, to eukaryotic cells. The bacteria can contain a prokaryotic expression cassette encoding the gene-editing cargo.

FIG. 2 is a series of illustrations (FIG. 2A, FIG. 2B, and FIG. 2C) of a plasmid system used to genetically engineer a bacterium to generate and deliver a gene-editing system in one or more embodiments of the present invention. FIG. 2A is a schematic representation with putative sequence lengths of the CRISPR-mediated gene editing plasmid (pSiCRISP) containing at least one prokaryotic promoter, an origin of replication, a selection marker, a crRNA leader, at least one spacer/crRNA homologous to a target DNA gene of interest, a tracr/tracrRNA, CRISPR direct repeat sequences, a nuclear localization signal (NLS), and a Cas gene. FIG. 2B is a schematic representation with putative sequence lengths of the invasion plasmid (pSiVEC) containing at least one prokaryotic promoter, an origin of replication, a selection marker, at least one invasion factor for receptor mediated endocytosis (Invasion factor A) and at least one invasion factor for endosomal escape (Invasion factor B). FIG. 2C is a schematic representation with putative sequence lengths of the CRISPR-mediated gene editing plasmid (pSiCRISP) containing at least one eukaryotic promoter, an origin of replication, a selection marker, a crRNA leader, at least one spacer/crRNA homologous to a target DNA gene of interest, a tracr/tracrRNA, CRISPR direct repeat sequences, a nuclear localization signal (NLS), and a Cas gene.

FIG. 3 is set of three images (FIG. 3A, FIG. 3B, and FIG. 3C) of FEC19 E. coli co-transformed with pSiCRISP_GFP and pSiVEC and PCR screened to validate the presence of pSiCRISP_GFP and pSiVEC components via PCR amplification of the (FIG. 3A) 424 bp NLS, (FIG. 3B) 727 bp Cas9, and (FIG. 3C) 201 bp inv gene expression cassette using specific primers for PCR.

FIG. 4 is a graph showing the proportion of A549 cells expressing GFP.

### DETAILED DESCRIPTION OF THE PREFERRED EMBODIMENT

Gene-editing is a form of genetic engineering in which DNA is inserted, deleted, modified or replaced in the genome of a living organism. The editing of specific sequences within a genome can be used for research purposes, as well as in gene therapy by targeting mutations to specific genes or inserting a functional gene into an organism and targeting it to replace the defective one that is associated with a pathogenic phenotype or genetic disease. The common approach to gene-editing is to use engineered or synthetic nucleases. One example is the CRISPR (clustered regularly interspaced short palindromic repeat) system which is rapidly advancing the field of genome editing.

CRISPR/CRISPR associated protein (Cas) has been employed to edit genomes of various model organisms such as bacteria, yeast, Caenorhabditis elegans, Drosophila melanogaster, plants, zebrafish, and mouse and human cells, (Mali et al. “Cas9 as a versatile tool for engineering biology,” Nat. Methods. 10:957-963 (2013). CRISPR/Cas systems have been delivered to these model organisms using a range of delivery mechanisms and vectors. More specifically CRISPR/Cas systems have been delivered to eukaryotic cells using a multitude of delivery methods including viral vectors and non-viral vectors.

Despite advancements in the field, efficient in vitro and in vivo delivery of gene-editing nuclease systems, including the CRISPR/Cas system, to targeted cells remains a challenge. There are a multitude of shortcomings associated with current CRISPR/Cas system delivery strategies that are hindering the clinical translation of gene-editing applications and therapies in human and animal medicine. We have developed a novel bacterial delivery platform for efficient delivery of any gene-editing nuclease system, including CRISPR/Cas systems, to targeted eukaryotic cells, providing cellular uptake and, where appropriate, nuclear translocation of the gene-editing nuclease system without host genome integration, for transient gene-editing applications.

The present invention provides a novel bacterial delivery platform for gene-editing systems, including CRISPR systems, to provide tissue specific delivery and intracellularization of genome editing components in any eukaryotic cell (dividing and non-dividing) without eukaryotic-cell genomic integration.

Gene-editing approaches are generally characterized by the use of a nuclease-based system (i.e. CRISPR/Cas) to effectively address a wide range of gene-editing applications including prevention of infectious disease, chronic non-infectious disease and other genetic disorders. Commonly, these nucleases create site-specific double-strand breaks at desired locations in the genome. The induced double-strand breaks are repaired through nonhomologous end-joining (NHEJ) or homologous recombination (HR), resulting in targeted mutations to the genome. An example of a nuclease-based system of gene editing is the CRISPR system. Other examples include, but are not limited to, transcription activator-like effector-based nucleases (TALENs), meganucleases, zinc finger nucleases (ZFNs), ARC nucleases, nucleases based on the Argonaute system, TtAgo nuclease systems, and other homing endonucleases.

The CRISPR system is comprised of two important components. The first component is a single guide RNA oligonucleotide (sgRNA) or guide RNA (gRNA) that represents the fusion of a CRISPR RNA (crRNA) with a tracrRNA. The second component is a Cas nuclease. These two components combined represent the sgRNA/gRNA and the CRISPR-associated protease/nuclease. When a protospacer-adjacent motif (PAM) is present in a genomic locus on a target DNA molecule, the Cas nuclease is directed by the gRNA to that PAM locus and a site-specific double-stranded break in the DNA occurs to induce a stable gene-editing event. This gene-editing event can result in permanent gene knock-out (gene-removal) or knock-in (gene insertion).

CRISPR/Cas systems are categorized into 3 main types: I, II and III. The mechanism of gene-editing, differs among the three types, although it is believed that all three have applications in eukaryotic cells. The most commonly used CRISPR/Cas system in eukaryotes is Type II, which requires a single Cas protein, namely Cas9. Similar to CRISPR/Cas, other gene-editing systems provide a desired genetic mutation or genome modification event.

These gene-editing nuclease proteins and gene editing systems have been difficult to deliver for clinical and research discovery applications. For example, due to the large size of the Cas9 protein, Cas9′s positive charge, and strong negative charge of the sgRNA, effective co-delivery of the Cas9/sgRNA complex to a host eukaryotic cell and across the cell membrane was difficult with prior systems. The Cas9, sgRNA, and Cas9/sgRNA complex cannot cross the lipid bilayer of a cell membrane without a delivery vehicle. One way to achieve CRISPR/Cas gene editing is to deliver the Cas9 protein with the sgRNA using a plasmid. The sgRNA along with the sgRNA scaffold is incorporated into the plasmid along with a Cas9 cassette. The plasmid can also include nuclear localization signals (NLS) for the gene-editing components being delivered to the nucleus. For example, the sgRNA and Cas9 protein may require a NLS to allow both to travel from the cytoplasm to the nucleus for transcription. This invention employs plasmids with prokaryotic promoters to drive expression of the gene-editing systems within the bacterial cell used for delivery to the eukaryotic cell. In this invention, the plasmids, which use prokaryotic promoters to drive expression of the gene-editing system (i.e. direct repeat sequences, tracr/tracrRNA, spacer, Cas9, and NLS), are transformed into the nonpathogenic bacterial cells. Additionally, the bacterial cells are engineered to be invasive, enabling them to enter the eukaryotic cells through receptor mediated endocytosis. The combined construct of the bacteria and plasmid(s) constitutes the bacterial mediated gene-editing delivery system to be delivered to a eukaryotic cell, along with gene-editing cargo expressed under the control of a prokaryotic promoter in certain embodiments.

The bacterial mediated gene-editing delivery platform taught herein offers numerous advantages when compared to other gene-editing delivery methods. First, the system facilitates transient delivery. The bacterial delivery platform achieves the desired editing event rapidly, minimizing off-target editing, increasing DNA specificity, and reducing any undesired consequences due to prolonged CRISPR/Cas or other nuclease editing systems in the cells.

Second, the system is non-immunogenic. The bacterial cells are engineered to evade antigen presenting cell recognition and the system does not induce immune or other cytokine responses in the host. In contrast, antibodies can be formed against other delivery vehicles like nanoparticles. And liposomes and viral vectors can induce an immune response.

Third, the system is non-integrating. In certain embodiments, expression of gene-editing components is exclusively derived from prokaryotic promoters. This inhibits host genome integration and provides controlled delivery of CRISPR/Cas or other nuclease editing systems and eliminates risk of carcinogenesis to the host and other unwanted side-effects. It is contemplated, however, that the system could employ plasmids with eukaryotic promoters, and use the nonpathogenic bacterium as a delivery vehicle for the plasmid, which would then be transcribed in the target cell. Fourth, the system is highly stable. The delivery platform is not inhibited by exposure to serum, proteases and nucleases, allowing the bacterial vehicles to remain stable until reaching their target site. Unlike other non-viral vectors, our bacterial delivery platform is not eliminated by phagocytic clearance, which further contributes to its stability.

Fifth, the system enables a large payload delivering capacity. The bacterial delivery platform is able to effectively deliver Cas enzymes such as Cas9, despite the large size of the enzymes. Other non-viral vectors struggle to deliver sufficient Cas9 concentrations to the target tissues but the system taught herein functions via receptor mediated endocytosis for effective intracellularization and endosomal release of the bacterial vehicles containing the gene-editing system being delivered.

Sixth, the system provides a large therapeutic window. The bacterial delivery platform can be administered at a range of dosages to accomplish a gene-editing effect without inducing toxicity.

Seventh, the system enables efficient delivery. The delivery platform can efficiently enter into eukaryotic cells through receptor mediated endocytosis and efficiently escape the host endosome to facilitate delivery of the gene-editing system.

To date, efficient in vivo delivery of gene-editing and CRISPR/Cas systems to targeted cells has been a challenge. Current strategies of CRISPR/Cas delivery are based primarily on mechanical approaches (electroporation, hydrodynamic injection, microinjection) and viral vector delivery (lentivirus, adenovirus, adenoassociated virus). Many of these methods cannot be easily translated for clinical application in an animal or human patient. Non-viral delivery methods, such as liposomes and nanoparticles, are also used. However, in order to attain therapeutic efficacy, the CRISPR/Cas system needs to be delivered more efficiently and specifically to avoid unwanted side effects and safety concerns due to systemic administration, trafficking to unwanted tissues, and host integration.

Although viral vectors have been used in the delivery of the CRISPR/Cas system in vitro and in vivo, their fundamental shortcomings, such as the risk of carcinogenesis due to host genomic integration, limited insertion size, stimulation of undesired immune responses, and difficulty in large-scale production, severely limit their further applications. Host genomic integration using viral vector delivery can result in high-level and prolonged expression of the Cas protein or other nucleases, contributing to higher off-target effects and strong immune responses in the host. Alternative non-viral delivery systems for CRISPR/Cas are urgently needed. Non-viral vectors represent another potential option for CRISPR/Cas delivery. These include but are not limited to liposomes, nanoparticles, exosomes, microvesicles, cell-penetrating peptides (CPPs), and other lipids, polymers, and lipid nanoparticles. Despite their utility in vitro, complex physiological conditions create barriers to using these non-viral delivery approaches in vivo.

The present invention provides a system and method to deliver gene-editing or CRISPR/Cas systems to eukaryotic cells using invasive, non-pathogenic bacteria containing prokaryotic expression cassettes to drive expression of those genes prior to delivery. In other words, with this invention bacteria are utilized that are both invasive and nonpathogenic, and that contain plasmids driven by prokaryotic promoters which confer the invasive properties of the bacteria, thereby allowing them to enter eukaryotic cells.

An advantage of using bacteria containing prokaryotic expression cassettes is that the necessary gene-editing cargo (oligonucleotides and nuclease proteins) are only expressed by the bacteria. They are generated by the bacteria prior to delivery. Once the bacterial cells are taken up and into the cytoplasm of the host eukaryotic cell, the plasmid containing the gene-editing genes and proteins is unable to be expressed in the eukaryotic cell due to polymerase restrictions. This prevents integration of these gene-editing genes and proteins and any other plasmid DNA into the host's genome. This is an advantage because genomic integration is associated with detrimental side-effects, including inability to control expression of these genes, carcinogenesis, and toxicity, among others (Kim S et al. 2014. Highly efficient RNA-guided genome editing in human cells via delivery of purified Cas9 ribonucleoproteins. Genome Research 24:1012-1019). The gene-editing event (targeted DNA modification) only needs to happen one time per copy of DNA to drive a permanent genetic modification through all cell populations. Therefore, in some instances, it is an advantage to use a delivery system for transient introduction of a gene-editing system in which the gene-editing components (i.e. Cas9 and sgRNA) are not integrated into the genome of the host cell. With integrative gene-editing approaches (i.e. viral vector delivery), the DNA mutation continues to occur in all copies of DNA because the gene-editing components (Cas9 and sgRNA) are integrated into the target cell genome. Avoiding integration into the target cell genome provides for controlled expression of the gene-editing components and further limits toxicity and off-target mutagenesis in the target cell.

The present invention employs bacteria that contain invasion factors (e.g. inv and hlyA) that allow the bacteria enter a eukaryotic cell and to deliver nuclease-based gene-editing cargo (e.g. CRISPR, Talens, zinc fingers are examples) to a eukaryotic cell. The use of bacteria that contain a plasmid with eukaryotic promoters, results in a system where it is the plasmid with the gene-editing system that is delivered and the gene-editing cargo is made inside the eukaryotic cell. A system employing prokaryotic promoters implicates the bacterium as the site of transcription and expression of the gene-editing system. Both routes of delivery (e.g. using prokaryotic promoters and eukaryotic promoters) are contemplated by the invention herein, but the system employing prokaryotic promoters offers some significant advantages, including ensuring that the system is non-integrating in the target cell.

The present invention will find a wide range of applications. For example, in diagnostics, to detect genome mutations associated with genetic disorders or to detect rejection markers prior to organ transplantation. The present invention can be used for the treatment of disease (infectious bacterial and viral and non-infectious) in which foreign DNA from an infectious agent present inside a eukaryotic cell is targeted for mutation, CAR-T applications for cancer treatment, prevention of disease in cases where a DNA modification is associated with a reduced infectious phenotype, treating genetic disorders in cases where a DNA mutation is associated with an unwanted genetic phenotype (i.e. cystic fibrosis, Huntington's disease, fragile X syndrome, sickle cell anemia, and familial hypercholesterolemia), and genome wide genetic screening in cases where it is important to identify functional genes or genetic sequences that influence a physiological effect.

The present invention further provides a method of chromosome painting in living cells. This system could be used to conduct CRISPR imaging as a method for detecting the chromatin dynamics in living cells. Using catalytically inactive dCas9 (nuclease dead Cas9 binds to DNA but cannot cleave the DNA to which its bound) fused to a fluorescent marker like green fluorescent protein (GFP), researchers have turned dCas9 into a customizable DNA labeler compatible with fluorescence microscopy in living cells. Alternatively, the CRISPR gRNA can be fused to protein-interacting RNA aptamers, which recruit specific RNA-binding proteins (RBPs) tagged with fluorescent proteins to visualize targeted genomic loci. Further applications include the ability to generate transgenic animals in which an animal is genetically modified to generate a target genotype or phenotype which for example is useful for scientific research applications such as the study of genetics and disease, or useful for generating for example agricultural animals that are resistant to a particular disease or agricultural species (plants) that are resistant to a particular disease or disorder.

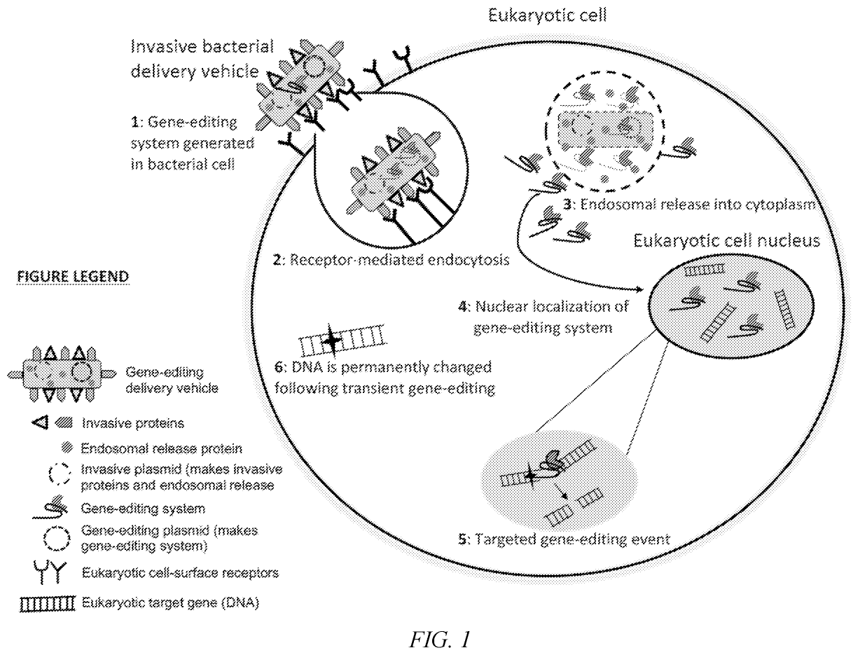

FIG. 1 provides a graphical representation of a bacterial mediated gene-editing delivery system using invasive, non-pathogenic bacteria to deliver gene-editing cargo, such as the CRISPR/Cas systems, to eukaryotic cells. In the exemplary system the bacteria contain a prokaryotic expression cassette encoding gene-editing cargo. A representation, provided by way of a non-limiting example, of the system's ability to generate and deliver CRISPR/Cas9 gene-editing cargo to a mammalian cell is depicted. The figure illustrates the operation of the bacterial mediated gene-editing delivery system as it proceeds through multiple steps in a process of delivering and effectuating the gene-editing of a eukaryotic cell.

First, a CRISPR associated protein 9 (Cas9) sequence, a trans-activating CRISPR RNA (tracrRNA), at least one CRISPR direct repeat sequence, a nuclear localization signal (NLS), and at least one spacer sequence, all part of the gene-editing plasmid, are transcribed within the bacterium (invasive bacterial delivery vehicle). Such transcription can occur prior to entry in a target eukaryotic cell. While inside the bacterium, the tracrRNA hybridizes to the direct repeats of crRNA and is then processed into mature single guide RNAs (sgRNAs) containing individual spacer sequences, while the Cas9 RNA transcript is translated into an active enzyme. These components (Cas9 enzyme and mature sgRNAs) make the CRISPR/Cas9 gene-editing system. The bacterium can optionally express the mc gene for RNase III either from a recombinant plasmid or the bacterial chromosome. Second, the bacterial vehicles generating the CRISPR/Cas9 gene-editing system are targeted to a mammalian cell by an invasion factor, such as the invasin protein, and taken into the cell by receptor mediated endocytosis (RME). Third, the endosome is broken down by a second invasion factor for endosomal release, such as LLO, releasing the sgRNA (mature crRNA:tracrRNA complex) and Cas9 enzyme for translocation to the eukaryotic cell nucleus, guided by the NLS. Fourth, once inside the nucleus, the sgRNA directs Cas9 to the DNA target consisting of the protospacer and the downstream protospacer adjacent motif (PAM) via heteroduplex formation between the crRNA and the protospacer DNA. Fifth, Cas9 mediates cleavage of the target DNA upstream of the PAM to create a double-strand break within the protospacer. In the sixth and final step, the target DNA is permanently modified. This graphic describes an example process for adapting this bacterial platform to generate, deliver, and direct CRISPR complex activity in the nuclei of eukaryotic cells for targeted gene-editing.

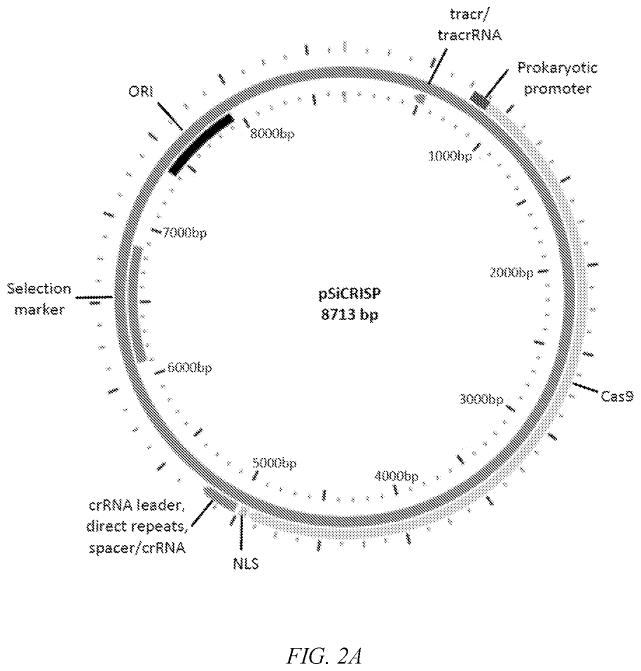

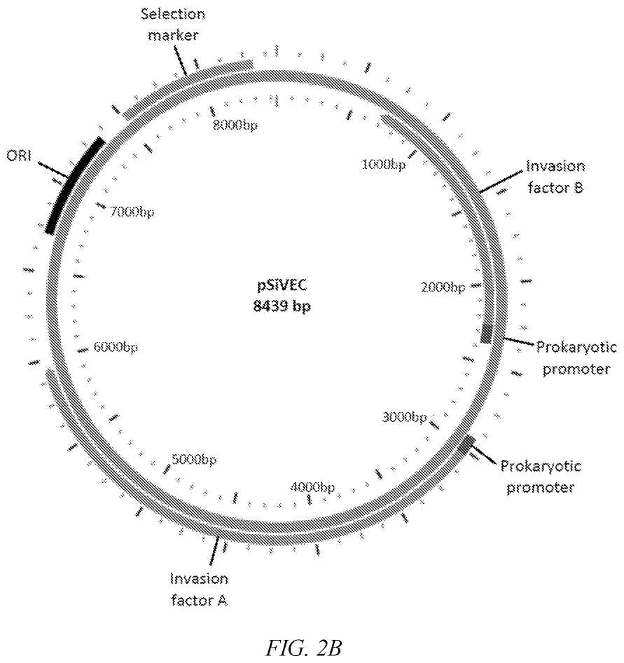

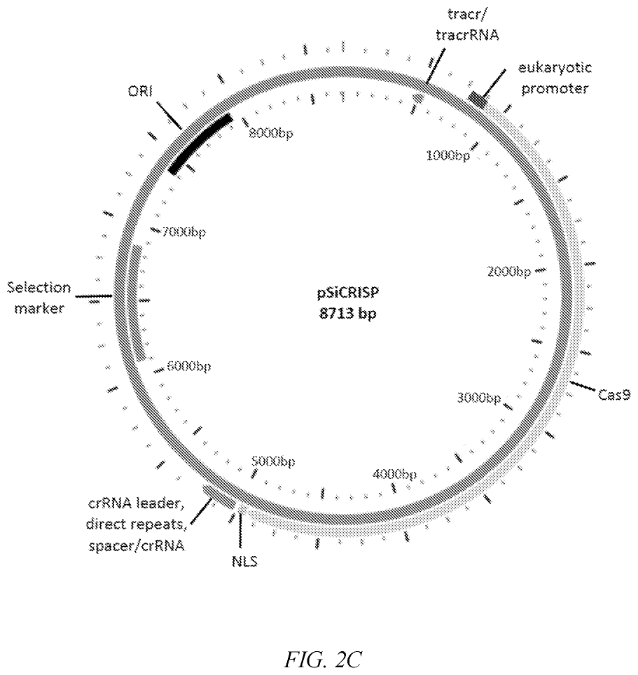

FIGS. 2A and 2B provide illustrations of a plasmid system used to genetically engineer a bacterium to generate and deliver a gene-editing system in one or more embodiments of the present invention. FIG. 2A is a schematic representation with putative sequence lengths of the CRISPR-mediated gene editing plasmid (pSiCRISP) containing at least one prokaryotic promoter, an origin of replication, a selection marker, a crRNA leader, at least one spacer/crRNA homologous to a target DNA gene of interest, a tracr/tracrRNA, CRISPR direct repeat sequences, a NLS, and a Cas gene. In addition to the pSiCRISP plasmid providing the gene editing system, this system requires components which permit the bacterium to be invasive to eukaryotic cells. FIG. 2B is a schematic representation with putative sequence lengths of the invasion plasmid (pSiVEC) containing at least one prokaryotic promoter, an origin of replication, a selection marker, at least one invasion factor for receptor mediated endocytosis (Invasion factor A) and at least one invasion factor for endosomal escape (Invasion factor B). These two plasmid systems can be co-transformed into the bacterium. In another embodiment of the invention, the components of these two plasmids may be combined into a single recombinant plasmid for bacterial transformation. In another embodiment of the invention, components of these two plasmids may be expressed from the chromosome of the bacterium.

The following examples are given for the purpose of illustrating various embodiments of the invention and are not meant to limit the present invention in any fashion. The following examples, along with the methods described herein are representative of preferred embodiments, are exemplary, and are not intended as limitations on the scope of the invention. Changes therein and other uses which are encompassed within the spirit of the invention as defined by the scope of the claims will occur to those skilled in the art.

### EXAMPLE 1

### Generation of Invasive Bacterium Comprising the pSiCRISP Plasmid Capable of Directing Gene-Editing Activity

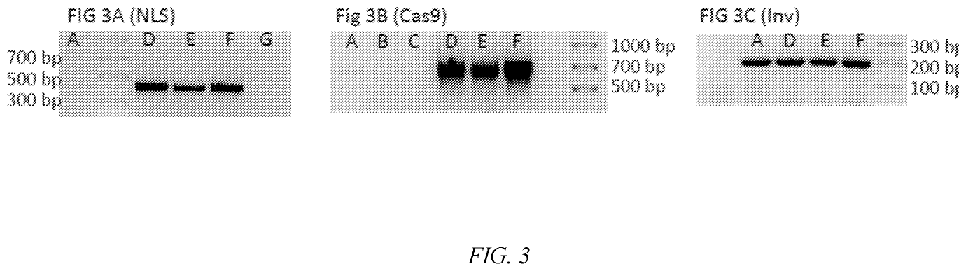

The pSiCRISP_GFP plasmid representing a CRISPR-complex directing plasmid expressing crRNA for green fluorescent protein (GFP) was synthesized and transformed into E. coli bacteria (FEC19). The FEC19 bacteria were additionally engineered to be invasive to eukaryotic cells via co-transformation with pSiVEC containing inv and hlyA genes for invasin-mediated receptor mediated endocytosis and LLO-mediated endosomal release, respectively. Resulting FEC19 colonies containing pSiCRISP-GFP and pSiVEC were plated onto Brain Heart Infusion (BHI) agar containing appropriate antibiotics for selection. Resulting FEC19 bacterial colonies (identified as A, B, C, D, E, F and G) were screened by PCR to demonstrate the successful transformation of the pSiCRISP and pSiVEC plasmids inside the FEC19 bacteria as a way to validate the composition of aspects of the present invention. The pSiCRISP_GFP sequences specific for the Cas9 gene and NLS, and the pSiVEC sequence specific for the inv gene were amplified using specific primers in PCR assays. Primers amplifying a 424 bp product (FIG. 3A) were used to verify presence of the NLS (screened in colonies A, D, E, F and G), primers amplifying a 727 bp product (FIG. 3B) were used to verify presence of the Cas9 gene (screened in colonies A, B, C, D, E and F), and primers amplifying a 201 bp product (FIG. 3C) were used to verify the presence of the inv gene (screened in colonies A, D, E and F). A 1 kb ladder is used in the figure(s) as a reference. Cas9 and the NLS sequences in FEC19+pSiCRISP_GFP (FEC19 transformed with pSiCRISP_GFP) were both present in colonies D, E and F. The inv sequence was present in all colonies tested. For each colony positive by PCR for inv and both Cas9 and the NLS (colonies D, E, and F), a single positive clone was sequence validated to confirm pSiCRISP_GFP and pSiVEC presence and propagated. This experiment demonstrates the generation of E. coli (FEC19) comprising a gene-editing plasmid (pSiCRISP) and further comprising a plasmid containing invasion factors for target cell entry (pSiVEC). Bacterial stocks for D, E and F were generated in BHI medium with proper antibiotics for selection and grown to an optical density measured at 600 nm wavelength (OD600) equal to 1.0. These FEC19+pSiCRISP_GFP+pSiVEC stocks (D, E, F) were frozen at −80° C. in 20% glycerol. A single frozen aliquot from each stock was thawed for plate enumeration. Briefly, a 1 mL aliquot was centrifuged for 5 min at 5000×g and the cells were resuspended in 1 mL of BHI. The resulting bacterial suspensions were serially diluted and plated in triplicate on BHI agar containing antibiotics. Colony counts at each dilution were averaged to calculate the overall colony forming units (CFU)/mL and represented a viable concentration for stocks of FEC19+pSiCRISP_GFP+pSiVEC. This system allowed a quantitated, live inoculum stock to be directly used in all future assays.

### EXAMPLE 2

### CRISPR-Mediated Gene-Editing with FEC19+pSiCRISP+pSiVEC in a Mammalian Cell Line

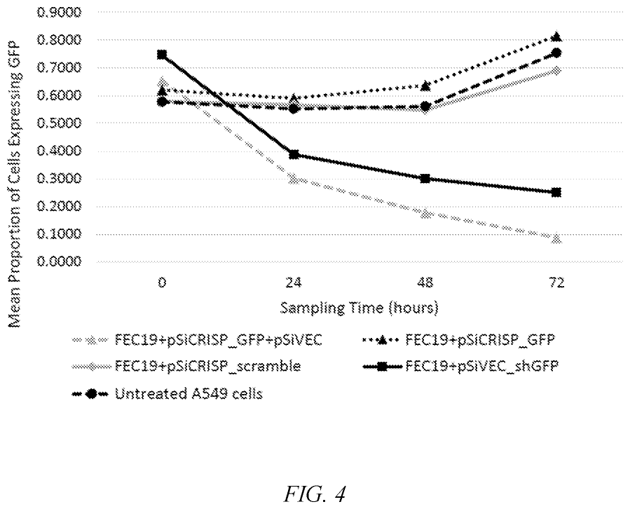

Human alveolar basal epithelial (A549) cells stably expressing GFP are maintained in Dulbecco's modified Eagle's Medium (DMEM) supplemented with 10% fetal bovine serum, 2 mM GlutaMAX, 100 U/mL penicillin, and 100 g/mL streptomycin at 37° C. with 5% CO₂ incubation. Invasive (transformed with pSiVEC) FEC19 bacteria containing the pSiCRISP_GFP plasmid are capable of entering A549 cells through RME and delivering the CRISPR-mediated gene-editing cargo necessary to knockout the gene of interest, GFP. This experiment includes a positive control treatment (FEC19+pSiVEC_shGFP) in which the pSiVEC plasmid is expressing a short hairpin RNA (shRNA) targeting GFP. This positive control treatment is included because knock-down of GFP using a shRNA targeting GFP in the A549 cells is expected and has been previously demonstrated. This invasion assay includes the following steps. First, the A549 cells are seeded at a fixed concentration into black 24-well plates. On the day of bacterial invasion, four FEC19 stocks are thawed: 1) FEC19+pSiCRISP_GFP+pSiVEC, 2) FEC19+pSiCRISP_GFP (non-invasive), 3) FEC19+pSiCRISP_scramble (expressing a scramble/nonsense crRNA)+pSiVEC, and 4) FEC19+pSiVEC_shGFP (expressing shRNA for GFP). The FEC19 bacteria stocks are serially diluted 1:10 in DMEM from 1×10⁷ CFU/mL to 1×10⁵ CFU/mL. A549 cells are incubated for 2 hours with 1 mL of each FEC19 treatment and subsequently rinsed with DMEM to remove unbound FEC19 bacteria. Using the Nexcelom Celigo, GFP expression is quantified over a time course of 72 hours with T0 readings being taken immediately before invasion. FIG. 4 demonstrates the proportion of A549 cells expressing GFP following incubation with 1×10⁷ CFU/mL of one of four FEC19 treatments at time 0, 24, 48, and 72 hours post treatment, compared to untreated A549 cells.

### GLOSSARY OF TERMS

All technical and scientific terms used herein have the same meaning as commonly understood by one of ordinary skill in the art to which this invention belongs, unless the technical or scientific term is defined differently herein.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi- stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double -stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single -stranded (such as sense or antisense) and double -stranded polynucleotides. The term “nucleic acid”, “polynucleotide” or “oligonucleotide” refers to a DNA molecule, an RNA molecule, or analogs thereof. As used herein, the terms “nucleic acid”, “polynucleotide” and “oligonucleotide” include, but are not limited to DNA molecules such as cDNA, genomic DNA or synthetic DNA and RNA molecules such as a guide RNA, messenger RNA or synthetic RNA. Moreover, as used herein, the terms “nucleic acid” and “polynucleotide” include single-stranded and double-stranded forms.

“Genomic DNA” refers to the DNA of a genome of an organism including, but not limited to, the DNA of the genome of a bacterium, virus, fungus, archaea, plant or animal.

“Manipulating” DNA encompasses binding, nicking one strand, or cleaving (i.e., cutting) both strands of the DNA, or encompasses modifying the DNA or a polypeptide associated with the DNA (e.g., amidation, methylation, etc.). Manipulating DNA can silence, activate, or modulate (either increase or decrease) the expression of an RNA or polypeptide encoded by the DNA.

“Gene-editing” refers to the process of changing the genetic information present in the genome of a cell. This gene-editing may be performed by manipulating genomic DNA, resulting in a modification of the genetic information. Such gene-editing may or may not influence expression of the DNA that has been edited.

As used herein, the term “guide RNA” generally refers to an RNA molecule (or a group of RNA molecules collectively) that can bind to a Cas protein and aid in targeting the Cas protein to a specific location within a target polynucleotide (e.g., a DNA). A guide RNA can comprise a crRNA segment and a tracrRNA segment. As used herein, the term “crRNA” or “crRNA segment” refers to an RNA molecule or portion thereof that includes a polynucleotide-targeting guide sequence, a stem sequence, and, optionally, a 5′-overhang sequence. As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The term “guide RNA” encompasses a single guide RNA (sgRNA), where the crRNA segment and the tracrRNA segment are located in the same RNA molecule. The term “guide RNA” also encompasses, collectively, a group of two or more RNA molecules, where the crRNA segment and the tracrRNA segment are located in separate RNA molecules. The term “modification” in the context of an oligonucleotide or polynucleotide includes but is not limited to (a) end modifications, e.g., 5′ end modifications or 3′ end modifications, (b) nucleobase (or “base”) modifications, including replacement, insertion, or removal of bases, (c) sugar modifications, including modifications at the 2′, 3′, and/or 4′ positions, and (d) backbone modifications, including modification or replacement of the phosphodiester linkages. The term “modified nucleotide” generally refers to a nucleotide having a modification to the chemical structure of one or more of the base, the sugar, and the phosphodiester linkage or backbone portions, including nucleotide phosphates.

As used more generally herein, “modification” refers to a chemical moiety, or portion of a chemical structure, which differs from that found in unmodified ribonucleotides, namely adenosine, guanosine, cytidine, and uridine ribonucleotides. The term “modification” may refer to type of modification. For example, “same modification” means same type of modification, and “the modified nucleotides are the same” means the modified nucleotides have the same type(s) of modification while the base (A, G, C, U, etc.) may be different. Similarly, an adaptor with “two modifications” is a guide RNA with two types of modifications, which may or may not be in the same nucleotide, and each type may appear in multiple nucleotides in the adaptor. Similarly, an adaptor polynucleotide with “three modifications” is an adaptor with three types of modifications, which may or may not be in the same nucleotide, and each, type may appear in multiple nucleotides.

A “donor polynucleotide” is a nucleotide polymer or oligomer intended for insertion at a target polynucleotide. The donor polynucleotide may be a natural or a modified polynucleotide, a RNA-DNA chimera, or a DNA fragment, either single- or double- stranded, or a PGR amplified ssDNA or dsDNA fragment. A fully double-stranded donor DNA is advantageous since it might provide an increased stability, since dsDNA fragments are generally more resistant than ssDNA to nuclease degradation. The terms “xA”, “xG”, “xC”, “xT”, or “x(A,G,C,T)” and “yA”, “yG”, “yC”, “yT”, or “y(A,G,C,T)” refer to nucleotides, nucleobases, or nucleobase analogs as described by Krueger et al., “Synthesis and Properties of Size-Expanded DNAs: Toward Designed, Functional Genetic Systems”, Acc. Chem. Res. 2007, 40, 141-150 (2007), the contents of which is hereby incorporated by reference in its entirety.

As used herein, the term “target polynucleotide” or “target” refers to a polynucleotide containing a target nucleic acid sequence: A target polynucleotide may be single-stranded or double-stranded, and, in certain embodiments, is double-stranded DNA. In certain embodiments, the target polynucleotide is single-stranded RNA. A “target nucleic acid sequence” or “target sequence,” as used herein, means a specific sequence or the complement thereof that one wishes to bind to, nick, or cleave using a CRISPR/Cas system or other gene-editing system.

The term “hybridization” or “hybridizing” refers to a process where completely or partially complementary polynucleotide strands come together under suitable hybridization conditions to form a double-stranded structure or region in which the two constituent strands are joined by hydrogen bonds. As used herein, the term “partial hybridization” includes where the double-stranded structure or region contains one or more bulges or mismatches. Although hydrogen bonds typically form between adenine and thymine or adenine and uracil (A and T or A and U) or cytosine and guanine (C and G), other noncanonical base pairs may form (See e.g., Adams el al., The Biochemistry of the Nucleic Acids, 11th ed., 1992). It is contemplated that modified nucleotides may form hydrogen bonds that allow or promote hybridization. By “hybridizable” or “complementary” or “substantially complementary” it is meant that a nucleic acid (e.g. RNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position a protein -binding segment (dsRNA duplex) of a guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 55%, at least 60%, at least 70%, at least 80%, at least 90%, at least 95%, at least 99%, or 100%) sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The term “cleavage” or “cleaving” refers to breaking of the covalent phosphodiester linkage in the ribosylphosphodiester backbone of a polynucleotide. The terms “cleavage” or “cleaving” encompass both single-stranded breaks and double-stranded breaks. Double-stranded cleavage can occur as a result of two distinct single-stranded cleavage events. Cleavage can result in the production of either blunt ends or staggered ends.

The term “CRISPR-associated protein” or “Cas protein” refers to a wild type Cas protein, a fragment thereof, or a mutant or variant thereof. The term “Cas mutant” or “Cas variant” refers to a protein or polypeptide derivative of a wild type Cas protein, e.g., a protein having one or more point mutations, insertions, deletions, truncations, a fusion protein, or a combination thereof. In certain embodiments, the “Cas mutant” or “Cas variant” substantially retains the nuclease activity of the Cas protein. In certain embodiments, the “Cas mutant” or “Cas variant” is mutated such that one or both nuclease domains are inactive. In certain embodiments, the “Cas mutant” or “Cas variant” has nuclease activity. In certain embodiments, the “Cas mutant” or “Cas variant” lacks some or all of the nuclease activity of its wild-type counterpart.

A synthetic guide RNA that has “gRNA functionality” is one that has one or more of the functions of naturally occurring guide RNA, such as associating with a Cas protein, or a function performed by the guide RNA in association with a Cas protein. In certain embodiments, the functionality includes binding a target polynucleotide. In certain embodiments, the functionality includes targeting a Cas protein or a gRNA:Cas protein complex to a target polynucleotide. In certain embodiments, the functionality includes nicking a target polynucleotide. In certain embodiments, the functionality includes acting within a gRNA:Cas system to cleave a target polynucleotide. In certain embodiments, the functionality includes associating with or binding to a Cas protein. In certain embodiments, the functionality is any other known function of a guide RNA in a CRISPR/Cas system with a Cas protein, including an artificial CRISPR/Cas system with an engineered Cas protein. In certain embodiments, the functionality is any other function of natural guide RNA. The synthetic guide RNA may have gRNA functionality to a greater or lesser extent than a naturally occurring guide RNA. In certain embodiments, a synthetic guide RNA may have greater functionality as to one property and lesser functionality as to another property.

As used herein, the term “segment” of a sequence refers to any portion of the sequence (e.g., a nucleotide subsequence or an amino acid subsequence) that is smaller than the complete sequence. Segments of polynucleotides can be any length, for example, at least 5, 10, 15, 20, 25, 30, 40, 50, 75, 100, 150, 200, 300 or 500 or more nucleotides in length. A portion of a guide sequence can be about 50%, 40%, 30%, 20%, 10% of the guide sequence, e.g., one-third of the guide sequence or shorter, e.g., 7, 6, 5, 4, 3, or 2 nucleotides in length.

The term “derived from” in the context of a molecule refers to a molecule isolated or made using a parent molecule or information from that parent molecule. For example, a Cas9 single mutant nickase and a Cas9 double mutant null-nuclease are derived from a wild-type Cas9 protein.

The term “substantially identical” in the context of two or more polynucleotides (or two or more polypeptides) refers to sequences or subsequences that have at least about 60%, at least about 70%, at least about 80%, at least about 90%, about 90-95%, at least about 95%, at least about 98%, at least about 99% or more nucleotide (or amino acid) sequence identity, when compared and aligned for maximum correspondence using a sequence comparison algorithm or by visual inspection. Preferably, the “substantial identity” between polynucleotides exists over a region of the polynucleotide at least about 50 nucleotides in length, at least about 100 nucleotides in length, at least about 200 nucleotides in length, at least about 300 nucleotides in length, at least about 500 nucleotides in length, or over the entire length of the polynucleotide. Preferably, the “substantial identity” between polypeptides exists over a region of the polypeptide at least about 50 amino acid residues in length, at least about 100 amino acid residues in length, or over the entire length of the polypeptide.

As disclosed herein, a number of ranges of values are provided. It is understood that each intervening value, to the tenth of the unit of the lower limit, between the upper and lower limits of that range is also specifically contemplated. Each smaller range or intervening value encompassed by a stated range is also specifically contemplated. The term “about” generally refers to plus or minus 10% of the indicated number. For example, “about 10%” may indicate a range of 9% to 11%, and “about 20” may mean from 18-22. Other meanings of “about” may be apparent from the context, such as rounding off, so, for example “about 1” may also mean from 0.5 to 1.4.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g. tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

As used herein, a “promoter sequence” is a DNA regulatory region capable of binding RNA polymerase and initiating transcription of a downstream (3′ direction) coding or non-coding sequence. For purposes of defining the present invention, the promoter sequence is bounded at its 3″ terminus by the transcription initiation site and extends upstream (5′ direction) to include the minimum number of bases or elements necessary to initiate transcription at levels detectable above background. Within the promoter sequence a transcription initiation site will be found, as well as protein binding domains responsible for the binding of RNA polymerase. Various promoters, including inducible promoters, may be used to drive the vectors as described in the present disclosure.

A promoter can be a constitutively active promoter (i.e., a promoter that is constitutively in an active (“ON”) state), it may be an inducible promoter (i.e., a promoter whose state, active (“ON”) or inactive (“OFF”), is controlled by an external stimulus, (e.g., the presence of a particular temperature, compound, or protein). Where in the prior art suitable promoters for the expression of the Cas proteins have been derived from viruses (which can therefore be referred to as viral promoters) also preferred are the promoters that are used to express the Cas9 protein in bacteria. It is also possible that a promoter that is known to drive Cas9 expression in one bacterium is used to drive the expression of a Cas9 protein derived from a different (species) of bacterium. In such case, it is said that said promoter is heterologous with respect to the Cas9 protein. The term “prokaryotic promoter” refers to promoters that are active within a bacterium for transcription of a sequence. The bacteria can be further engineered to express a phage RNA polymerase, thus facilitating use of a phage promoter. In certain aspects, the use of a phage promoters can beneficial to drive bacteria to express toxic genes. For example, using a phage promoter and placing it upstream of a toxic gene can enable, or force, force the bacteria to make a large, and perhaps toxic, quantities of a gene-editing nuclease for example.

### Invasive Microorganisms

As used herein, the term “invasive” when referring to a microorganism, a bacterium or bacterial therapeutic particle (BTP), refers to a microorganism that is capable of delivering at least one molecule, e.g , an RNA or RNA-encoding DNA molecule, to a target cell. An invasive microorganism can be a microorganism that is capable of traversing a cell membrane, thereby entering the cytoplasm of said cell, and delivering at least some of its content, e.g., RNA or RNA-encoding DNA, into the target cell. The process of delivery of the at least one molecule into the target cell preferably does not significantly modify the invasion apparatus.

As used herein, the term “transkingdom” refers to a delivery system that uses bacteria (or another invasive microorganism) to generate nucleic acids and deliver the nucleic acids intracellularly (i.e. across kingdoms: prokaryotic to eukaryotic, or across phyla: invertebrate to vertebrate) within target tissues for processing without host genomic integration.

Invasive microorganisms include microorganisms that are naturally capable of delivering at least one molecule to a target cell, such as by traversing the cell membrane, e.g., a eukaryotic cell membrane, and entering the cytoplasm, as well as microorganisms which are not naturally invasive and which have been modified, e.g., genetically modified, to be invasive. In another preferred embodiment, a microorganism that is not naturally invasive can be modified to become invasive by linking the bacterium or BTP to an “invasion factor”, also termed “entry factor” or “cytoplasm-targeting factor”. As used herein, an “invasion factor” is a factor, e.g., a protein or a group of proteins which, when expressed by a non-invasive bacterium or BTP, render the bacterium or BTP invasive. As used herein, an “invasion factor” is encoded by a “cytoplasm-targeting gene”. Invasive microorganisms have been generally described in the art, for example, U.S. Pat. Pub. Nos. US 20100189691 A1 and US20100092438 A1 and Xiang, S. et al., Nature Biotechnology 24, 697-702 (2006). Each of which is incorporated by reference in its entirety for all purposes.

In a preferred embodiment the invasive microorganism is E. coli, as taught in the examples of the present application. However, it is contemplated that additional microorganisms could potentially be adapted to perform as transkingdom delivery vehicles for the delivery of gene-editing cargo. These non-virulent and invasive bacteria and BTPS would exhibit invasive properties, or would be modified to exhibit invasive properties, and may enter a host cell through various mechanisms. In contrast to uptake of bacteria or BTPs by professional phagocytes, which normally results in the destruction of the bacterium or BTP within a specialized lysosome, invasive bacteria or BTP strains have the ability to invade non-phagocytic host cells. Naturally occurring examples of such intracellular bacteria are Yersinia, Rickettsia, Legionella, Brucella, Mycobacterium, Helicobacter, Coxiella, Chlamydia, Neisseria, Burkolderia, Bordetella, Borrelia, Listeria, Shigella, Salmonella, Staphylococcus, Streptococcus, Porphyromonas, Treponema, and Vibrio, but this property can also be transferred to other bacteria or BTPs such as E. coli, Lactobacillus, Lactococcus, or Bifidobacteriae, including probiotics through the transfer of invasion-related genes (P. Courvalin, S. Goussard, C. Grillot-Courvalin, C.R. Acad. Sci. Paris 318, 1207 (1995)). Factors to be considered or addressed when evaluating additional bacterial species as candidates for use as transkingdom delivery vehicles include the pathogenicity, or lack thereof, of the candidate, the tropism of the candidate bacteria for the target cell, or, alternatively, the degree to which the bacteria can be engineered to deliver gene-editing cargo to the interior of a target cell, and any synergistic value that the candidate bacteria might provide by triggering the host's innate immunity.

A colony is defined as a visible mass of microorganisms all originating from a single mother cell, therefore a colony constitutes a clone of bacteria all genetically alike.

### Promoters

Among the gene expression regulatory elements, the promoter plays a central role. Along the promoter molecule, the transcription machinery is assembled and transcription is initiated. This early step is often rate-limiting relative to subsequent stages of protein production. Transcription initiation at the promoter may be regulated in several ways. For example, a promoter may be induced by the presence of a particular compound or external stimuli, express a gene during a specific stage of development, or constitutively express a gene. Thus, transcription of a transgene may be regulated by operably linking the coding sequence to promoters with different regulatory characteristics. Accordingly, a regulatory element such as a promoter, plays a pivotal role in enhancing the value of a transgenic organism.

As used herein, the term “promoter” refers to a polynucleotide molecule that is involved in recognition and binding of RNA polymerase and other proteins such as associated sigma factor and an activator protein to initiate transcription of an operably linked gene. A promoter may be isolated from the 5′ untranslated region (5′ UTR) of a genomic copy of a gene. Alternately, a promoter molecule may be artificially and synthetically produced or comprise modified DNA sequence. A promoter can be used as a regulatory element for modulating expression of an operably linked transcribable polynucleotide molecule. Promoters may themselves contain sub-elements such as cis-elements or enhancer domains that effect the transcription of operably linked genes.

In prokaryotes, the promoter consists of two short sequences at −10 and −35 positions upstream from the transcription start site. Sigma factors not only help in enhancing RNAP binding to the promoter but helps RNAP target which genes to transcribe. The sequence at −10 is called the Pribnow box, or the −10 element, and usually consists of the six nucleotides TATAAT. The Pribnow box is absolutely essential to start transcription in prokaryotes. The other sequence at −35 (the −35 element) usually consists of the six nucleotides TTGACA. Its presence allows a very high transcription rate. Both of the above consensus sequences, while conserved on average, are not found intact in most promoters. On average only 3 of the 6 base pairs in each consensus sequence is found in any given promoter. It should be noted that the above promoter sequences are only recognized by the sigma-70 protein that interacts with the prokaryotic RNA polymerase. Complexes of prokaryotic RNA polymerase with other sigma factors recognize totally different core promoter sequences.

Many regulatory elements act in cis (“cis elements”) and are believed to affect DNA topology, producing local conformations that selectively allow or restrict access of RNA polymerase to the DNA template or that facilitate selective opening of the double helix at the site of transcriptional initiation. Cis elements occur within the 5′ UTR associated with a particular coding sequence, and are often found within promoters and promoter modulating sequences (inducible elements). Cis elements can be identified using known cis elements as a target sequence or target motif using the BLAST programs. Examples of cis-acting elements in the 5′UTR associated with a polynucleotide coding sequence include, but are not limited to, promoters and enhancers.

In prokaryotes, the mRNA translation starts with UTG, GTG or in rare case UUG, which is usually preceded by sequences characteristic of a ribosomal binding site (RBS; Shine and Dalgarno, PNAS 71:1342-1346, 1974). The RBS is AG rich and usually is found 6-12 bp before initiation codon. The RBS is believed to be necessary for efficient mRNA translation in bacteria.

At least two types of information are useful in predicting promoter regions within a genomic DNA sequence. First, promoters may be identified on the basis of their sequence “content,” such as transcription factor binding sites and various known promoter motifs. (Stormo, Genome Research 10: 394-397, 2000). Such signals may be identified by computer programs that identify sites associated with promoters, such as TATA boxes and transcription factor (TF) binding sites. Second, promoters may be identified on the basis of their “location,” i.e. their proximity to a known or suspected coding sequence (Stormo). Prokaryotic promoters are typically found within a region of DNA extending approximately 1-500 basepairs in the 5′ direction from the transcriptional or translational start codon of a coding sequence. Thus, promoter regions may be identified by locating the translational start codon of a coding sequence or the transcriptional start site, and moving beyond the start codon in the 5′ direction to locate the promoter region.

Promoter sequence may be analyzed for the presence of common promoter sequence characteristics, such as a TATA-box and other known transcription factor binding site motifs. These motifs are not always found in every known promoter, nor are they necessary for every promoter to function, but when present, do indicate that a segment of DNA is a promoter sequence.

The activity or strength of a promoter may be measured in terms of the amount of mRNA tRNA, dsRNA, miRNA, rRNA, or protein is specifically accumulated during a particular period of time in the growth of a cell containing the transgene. An enhanced level of mRNA production may be required to produce a protein of commercial importance, or a protein for catalyzing a reaction to produce a compound of commercial importance. Coding sequences for the commercially important proteins can be identified by those skilled in the art and can be used with the promoters of the invention to effect production of mRNA and protein levels. The activity or strength of a promoter can also be measured in terms of enhanced cell growth when a cell is grown under selection pressure after it has been transformed with, for instance, a gene that allows for detoxification of a compound under the control of the promoter. Hybrid promoters are defined as a promoter that is generated by engineering bacteria or an expression cassette on a plasmid having the -35 region of one promoter and the -10 region of another promoter.

### Selectable Markers

As used herein the term “marker” refers to any transcribable polynucleotide molecule whose expression, or lack thereof, can be screened for or scored in some way. Marker genes for use in the practice of the present invention include, but are not limited to transcribable polynucleotide molecules encoding β-glucuronidase (GUS described in U.S. Pat. No. 5,599,670, which is incorporated herein by reference), green fluorescent protein and variants thereof (GFP described in U.S. Pat. Nos. 5,491,084 and 6,146,826, RFP and the like, all of which are incorporated herein by reference), or proteins that confer antibiotic resistance.

Useful antibiotic resistance markers, including those encoding proteins conferring resistance to kanamycin (nptII), hygromycin B (aph IV), streptomycin or spectinomycin (aad, spec/strep), and gentamycin (aac3 and aacC4) are known in the art, among others. An advantageous positive selection marker is hisD (histidinol dehydrogenase). The hisD gene of Salmonella typhimurium encodes histidinol dehydrogenase, which catalyzes the two-step NAD'-dependent oxidation of L-histidinol to L-histidine. In medium lacking histidine and containing histidinol, only cells expressing the hisD product survive.

Included within the term “selectable markers” are also genes which encode a secretable marker whose secretion can be detected as a means of identifying or selecting for transformed cells. Examples include markers that encode a secretable enzymes which can be detected catalytically. Other possible selectable marker genes will be apparent to those of skill in the art.

In specific embodiments, a selectable marker use may be hisD, green fluorescent protein (GFP), neomycin phosphotransferase II (nptII), luciferase (LUX), or an antibiotic resistance coding sequence (aadA). In certain embodiments, the selectable marker is a kanamycin, streptomycin and/or spectinomycin resistance marker. Examples of coding sequences providing tolerance to antibiotics and herbicides can be found, for instance, in US Patent Application Publications 20080305952 and 20080280361, which are incorporated herein by reference.

### Cell Transformation

The term “transformation” refers to the introduction of nucleic acid into a recipient host. The term “host” refers to prokaryotic cells, especially bacterial cells. As used herein, the term “transformed” refers to a cell or organism into which has been introduced a foreign polynucleotide molecule, such as a construct. The introduced polynucleotide molecule may be integrated into the genomic DNA of the recipient cell or organism such that the introduced polynucleotide molecule is inherited by subsequent progeny or may stay in cytoplasm as a self-replicating unit. A “transgenic” or “transformed” cell or organism also includes progeny of the cell or organism. The term “transgenic” refers to a cell or other organism containing one or more heterologous polynucleic acid molecules.

There are many methods for introducing heterologous polynucleic acid molecules into cells. The method generally comprises the steps of selecting a suitable host cell, transforming the host cell with a recombinant vector, and obtaining the transformed host cell. Suitable methods for introducing heterologous polynucleic acid molecules into prokaryotes include Freeze-thaw (Heat shock), triparental mating, and electroporation, among others. These methods are known to those skilled in the art of prokaryotic transformation.

### Kits

Kits for practicing the methods of the invention are further provided. By “kit” is intended any manufacture (e.g., a package or a container) comprising at least one reagent, e.g., a gene editing bacterium of the invention. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

The term “administration” and variants thereof (e.g., “administering” a compound) in reference to a compound of the invention means introducing the compound into the system of the subject in need of treatment. When a compound of the invention is provided in combination with one or more other active agents (e.g., an AIV vaccine, etc.), “administration” and its variants are each understood to include concurrent and sequential introduction of the compound and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to a viral infection, an effective amount comprises an amount sufficient to prevent contracting the disease or to reduce the severity of the disease as evidenced by clinical disease, clinical symptoms, viral titer or virus shedding from the subject, or as evidenced by the ability to prevent or reduce transmission between animals. In some embodiments, an effective amount is an amount sufficient to delay onset of clinical illness and/or symptoms or to prevent the disease. In some embodiments, an effective amount is an amount sufficient to lower viral titers and/or reduce viral shedding. An effective amount can be administered in one or more doses.

As used herein, “treatment” refers to obtaining beneficial or desired clinical results. Beneficial or desired clinical results include, but are not limited to, any one or more of: alleviation of one or more symptoms, diminishment of extent of viral infection, stabilized (i.e., not worsening) state of viral infection, preventing or delaying spread (e.g., shedding) of the viral infection, preventing, delaying or slowing of viral infection progression, and/or maintain weight/weight gain. The methods of the invention contemplate any one or more of these aspects of treatment.

A “pharmaceutically acceptable” component is one that is suitable for use with humans and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof. The term “and/or” wherever used herein includes the meaning of “and”, “or” and “all or any other combination of the elements connected by said term”.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense. It is also to be understood that the following claims are intended to cover all of the generic and specific features of the invention herein described, and all statements of the scope of the invention which, as a matter of language, might be said to fall therebetween. Now that the invention has been described,

1-121. (canceled)

122. A composition for the delivery of a gene-editing system to a eukaryotic cell comprising a bacterium engineered to express an invasion factor to facilitate entry of the bacterium into a eukaryotic cell, a gene editing nuclease, and a guide RNA.

123. The composition for the delivery of a gene-editing system to a eukaryotic cell according to claim 122 wherein the gene editing nuclease is a CRISPR-associated (Cas) enzyme, an effector nuclease, a Tal-effector nuclease (TALEN), a transcription activator-like effectors (TALEs), an ARC nuclease, a zinc finger nuclease, a nuclease based on the Argonaute system, a TtAgo nuclease system or a combination thereof.

124. The composition for the delivery of a gene-editing system to a eukaryotic cell according to claim 123 wherein the gene editing nuclease is a CRISPR-associated (Cas) enzyme and the CRISPR-associated (Cas) enzyme is encoded by a Cas9 gene.

125. The composition for the delivery of a gene-editing system to a eukaryotic cell according to claim 122 wherein the guide RNA is a single guide RNA (sgRNA) comprising CRISPR direct repeat regions, spacer sequences, tracr sequences, crRNAs, or any portion or combination thereof.

126. The composition for the delivery of a gene-editing system to a eukaryotic cell according to claim 122 wherein the invasion factor, the gene editing nuclease, or the guide RNA is expressed from the chromosome of the bacterium.

127. The composition for the delivery of a gene-editing system to a eukaryotic cell according to claim 122 wherein at least two of the invasion factor, the gene editing nuclease, and the guide RNA are expressed from the chromosome of the bacterium.

128. The composition for the delivery of a gene-editing system to a eukaryotic cell according to claim 122 wherein the gene editing nuclease has a nuclear localization signal (NLS).

129. The composition for the delivery of a gene-editing system to a eukaryotic cell according to claim 122 wherein the bacterium comprises a plasmid encoding the gene editing nuclease and the guide RNA and wherein transcription of the gene editing nuclease and the guide RNA is under the control of a prokaryotic promoter.

130. The bacterium for delivery of a gene-editing system to a eukaryotic cell according to claim 129 wherein the plasmid encodes a nuclear localization signal.

131. The composition for the delivery of a gene-editing system to a eukaryotic cell according to claim 129 wherein the plasmid encodes a gene editing nuclease with a nuclear localization signal.

132. The composition for the delivery of a gene-editing system to a eukaryotic cell according to claim 129 wherein the prokaryotic promoter is a promoter selected from the group consisting of T7, lacUV5, gapA, T5, recA, Ptac, Patac, pA1, lac, Sp6, araBad, and trp.

133. The composition for the delivery of a gene-editing system to a eukaryotic cell according to claim 129 wherein the prokaryotic promoter is a hybrid or synthetic prokaryotic promoter.

134. The composition for the delivery of a gene-editing system to a eukaryotic cell according to claim 122 wherein the invasion factor is encoded by a gene selected from the group consisting of inv, hlyA, HA-1, hlyE and combinations thereof

135. The composition for the delivery of a gene-editing system to a eukaryotic cell according to claim 122 wherein the bacterium is a bacterium selected from the group consisting of Clostridium difficile, Escherichia coli, Clostridium tetani, Helicobacter pylori, Fusobacterium nucleatum, Gardnerella vaginitis, Porphyromonas gingivalis, Aggregatibacter actinomycetemcomitans, Listeria monocytogenes, Staphylococcus aureus, Campylobacter jejuni, Vibrio vulnificus, Salmonella typhi, Clostridium botulinum, Mycobacterium tuberculosis, Mycobacterium leprae, Mycobacterium lepromatosis, Corynebacterium diptheriae, Klebsiella pneumoniae, Acinetobacter baumannii, Streptococcus mutans, group B streptococci, Staphylococcus aureus, Streptococcus agalactiae, Streptococcus pneumonia, Enterococcus spp., Enterococcus faecalis, Listeria, Yersinia, Rickettsia, Shigella, E. coli, Salmonella spp., Legionella, Chlamydia, Brucella, Neisseria, Burkolderia, Bordetella, Borrelia, Coxiella, Mycobacterium, Helicobacter, Staphylococcus, Streptococcus, Porphyromonas, Vibrio, Treponema, Lactobacillus, and Bifidobacteriae.

136. A composition for the delivery of a gene-editing system to a eukaryotic cell comprising a bacterium engineered to express an invasion factor to facilitate entry of the bacterium into a eukaryotic cell, a CRISPR-associated (Cas) enzyme, and a single guide RNA (sgRNA) molecule.

137. The composition for the delivery of a gene-editing system to a eukaryotic cell according to claim 136 wherein the CRISPR-associated (Cas) enzyme has a nuclear localization signal (NLS).

138. The composition for the delivery of a gene-editing system to a eukaryotic cell according to claim 136 wherein the invasion factor, the gene editing nuclease, or the guide RNA is expressed from the chromosome of the bacterium.

139. The composition for the delivery of a gene-editing system to a eukaryotic cell according to claim 136 wherein at least two of the invasion factor, the gene editing nuclease, and the guide RNA are expressed from the chromosome of the bacterium.

140. The composition for the delivery of a gene-editing system to a eukaryotic cell according to claim 136 wherein the bacterium comprises a plasmid encoding the gene editing nuclease and the guide RNA and wherein transcription of the gene editing nuclease and the guide RNA is under the control of a prokaryotic promoter.

141. The composition for the delivery of a gene-editing system to a eukaryotic cell according to claim 140 wherein the prokaryotic promoter is a hybrid or synthetic prokaryotic promoter.

142. The composition for the delivery of a gene-editing system to a eukaryotic cell according to claim 136 wherein the invasion factor is encoded by a gene selected from the group consisting of inv, hlyA, HA-1, hlyE and combinations thereof

143. A composition for the delivery of a gene-editing system to a eukaryotic cell comprising a bacterium engineered to express an invasion factor and comprising a plasmid encoding a gene editing nuclease and a guide RNA.

144. The composition for the delivery of a gene-editing system to a eukaryotic cell according to claim 143 wherein the gene editing nuclease is a CRISPR-associated (Cas) enzyme and the CRISPR-associated (Cas) enzyme has a nuclear localization signal (NLS).

145. The composition for the delivery of a gene-editing system to a eukaryotic cell according to claim 143 wherein the plasmid comprises a eukaryotic promoter to control transcription of the gene editing nuclease and the guide RNA.

146. The composition for the delivery of a gene-editing system to a eukaryotic cell according to claim 145 wherein the eukaryotic promoter is a promoter selected from the group consisting of CMV, EFla, CAG, PGK, TRE, and U6 promoter.

147. A method of delivering a gene editing system to a target eukaryotic cell comprising the steps of:
providing a composition having a bacterium according to claim 136; and
contacting a target eukaryotic cell with the provided composition under conditions effective to allow entry of the bacterium into the target cell.

148. A method of delivering a gene editing system to a target eukaryotic cell comprising the steps of:
providing a composition having a bacterium according to claim 122; and
contacting the target eukaryotic cell with the provided composition under conditions effective to allow entry of the bacterium into the target cell.

149. The method of delivering a gene editing system to a target eukaryotic cell according to claim 148 wherein the invasion factor is encoded by a gene selected from the group consisting of an inv gene, a hlyA gene, an HA1 gene, a hlyE gene, and combinations thereof.

150. A method of delivering a gene editing system to a target eukaryotic cell comprising the steps of:
providing a composition having a bacterium according to claim 145; and
contacting the target eukaryotic cell with the provided composition under conditions effective to allow entry of the bacterium into the target cell.
